(19) United States
(12) Patent Application Publication
(10) Pub. No.: US 2026/0337164 A1
(43) Pub. Date: Oct. 29, 2026

(54) PROCESS AND INTERMEDIATES FOR THE PREPARATION OF A P2X3 INHIBITOR

(71) Applicant: CHIESI FARMACEUTICI S.P.A., Parma (IT)

(72) Inventors: Monica BOCCHI, Parma (IT); Luca CARDINALI, Parma (IT); Fausto PIVETTI, Parma (IT); Massimiliano MARI, Parma (IT); Paolo RONCHI, Parma (IT); David C. LATHBURY, Parma (IT); Edoardo MARIANI, Parma (IT)

(73) Assignee: CHIESI FARMACEUTICI S.P.A., Parma (IT)

(21) Appl. No.: 19/486,127

(22) PCT Filed: May 21, 2024
(86) PCT No.: PCT/EP2024/063879

§ 371 Date: Nov. 20, 2025

(30) Foreign Application Priority Data
May 22, 2023 (EP) 23174500.1

(51) Int. Cl. C07D 213/61; C07D 413/14

(52) U.S. Cl. CPC C07D 213/61; C07D 413/14

(57) ABSTRACT

The present invention relates to a process for the preparation of a P2X₃ inhibitor, i.e. (R)-6-(5-fluoropyridin-2-yl)-8-methoxy-N-(1-(5-methyl-1,2,4-oxadiazol-3-yl)ethyl)quinazolin-4-amine, or a pharmaceutically acceptable salt thereof. It also relates to intermediate compounds which are useful in such process and their preparation thereof. The synthesized P2X₃ inhibitor is suitable for medical use in pharmaceutical applications.

### FIELD OF THE INVENTION

The present invention relates to a process for the preparation of a P2X₃ inhibitor, i.e. (R)-6-(5-fluoropyridin-2-yl)-8-methoxy-N-(1-(5-methyl-1,2,4-oxadiazol-3-yl)ethyl)quinazolin-4-amine, or a pharmaceutically acceptable salt thereof. The present invention also relates to intermediate compounds which are useful in such process and their preparation thereof. The synthesized P2X₃ inhibitor is suitable for use in pharmaceutical applications, for instance in the treatment of respiratory diseases.

### BACKGROUND OF THE INVENTION

P2X receptors are cell surface ion channels activated by extracellular Adenosine 5-TriPhosphate (ATP). P2X receptor family comprises seven distinct subunit subtypes (P2X1-7) that assemble as homomeric and heteromeric channels. P2X₃ receptor is a member of this family and has been identified as a therapeutic target for the treatment of chronic cough (Ford A P, Undem B J: The therapeutic promise of ATP antagonism at P2X₃ receptors in respiratory and urological disorders, Front Cell Neurosci, December 19; 7:267, 2013). P2X₃ antagonists have been disclosed for instance in WO2016088838, WO2016091776, WO2020239953, WO2022112490, WO2022112491 and WO2022112493.

(R)-6-(5-fluoropyridin-2-yl)-8-methoxy-N-(1-(5-methyl-1,2,4-oxadiazol-3-yl)ethyl) quinazolin-4-amine (compound 1) is a potent P2X₃ antagonist which has been disclosed in WO2020239951 and has the following structural formula:

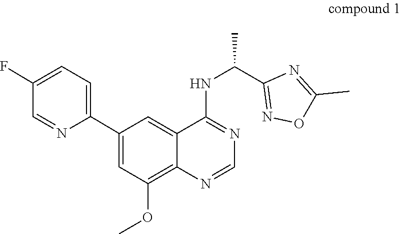 [SMILES: COc1cc(-c2ccc(F)cn2)cc2c(N[C@H](C)c3noc(C)n3)ncnc12]

WO2020239951 also disclosed a preparation of compound 1 according to the following:

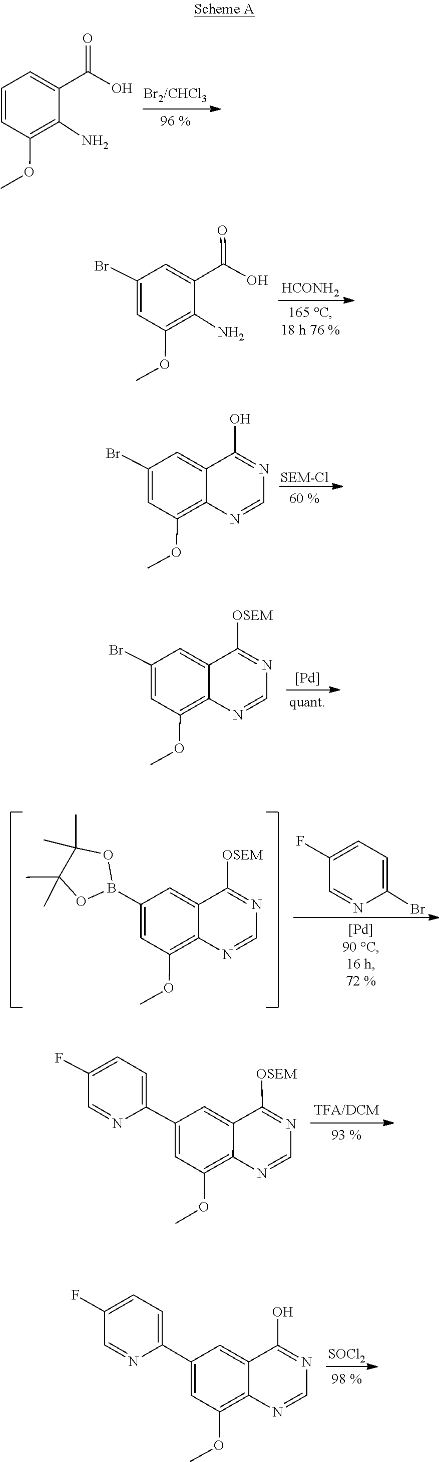

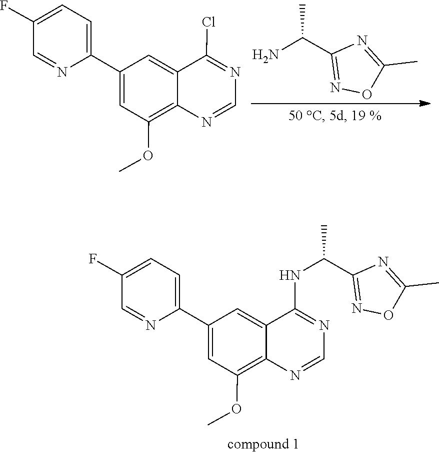

The process depicted in Scheme A starts from 2-amino-5-bromo-3-methoxybenzoic acid hydrobromide and leads to the target compound 1 in 8 steps with an overall yield of approximately 5.5%. Such process is lengthy and highly inefficient, as it has several disadvantages. In particular:
1. it requires the protection of the phenolic hydroxy group to avoid a competing reaction during the palladium-mediated Miyaura-Suzuki coupling involving 2-bromo-5-fluoropyridine, which increases the number of steps and decreases the overall yield;
2. it involves the formation at an early stage of the quinazoline bicyclic core which generates several bicyclic intermediate compounds which are poorly soluble;
3. it involves a lengthy and low-yielding final coupling step between (R)-1-(5-methyl-1,2,4-oxadiazol-3-yl)ethan-1-amine (compound 4) and the 4-chloro-quinazoline derivative, which takes as much as 5 days with a reported yield of 19%.

As (R)-6-(5-fluoropyridin-2-yl)-8-methoxy-N-(1-(5-methyl-1,2,4-oxadiazol-3-yl)ethyl) quinazolin-4-amine (compound 1) has been considered for drug development, there was a need to develop an efficient, simple and industrially viable synthetic route, which could overcome or at least lessen the drawbacks of the prior art method. It was of particular importance to devise an improved process that could achieve an increase in the overall yield with a reduced number of steps and that was suitable for scale-up. Such a process has been surprisingly developed and it is herein described, together with novel intermediates and preparations thereof.

### SUMMARY OF THE INVENTION

In a first aspect, the present invention relates to novel process intermediate compounds which are useful in the preparation of (R)-6-(5-fluoropyridin-2-yl)-8-methoxy-N-(1-(5-methyl-1,2,4-oxadiazol-3-yl)ethyl)quinazolin-4-amine (compound 1).

The invention provides 2-amino-5-(5-fluoropyridin-2-yl)-3-methoxybenzonitrile (intermediate 2)

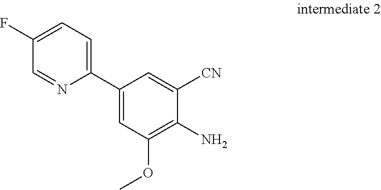 [SMILES: COc1cc(-c2ccc(F)cn2)cc(C#N)c1N]

or a pharmaceutically acceptable salt thereof.

The invention further provides (E)-N′-(2-cyano-4-(5-fluoropyridin-2-yl)-6-methoxyphenyl)-N,N-dimethylformimidamide (intermediate 3)

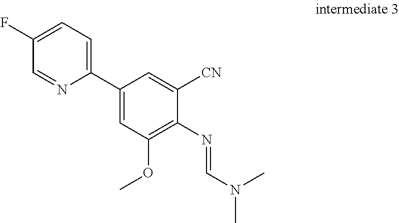 [SMILES: COc1cc(-c2ccc(F)cn2)cc(C#N)c1/N=C/N(C)C]

or a pharmaceutically acceptable salt thereof.

In a second aspect, the present invention relates to the use of intermediate 2 and/or intermediate 3 for the preparation of compound 1.

In a third aspect, the present invention relates to a process for the preparation of compound 1, or a pharmaceutically acceptable salt thereof:

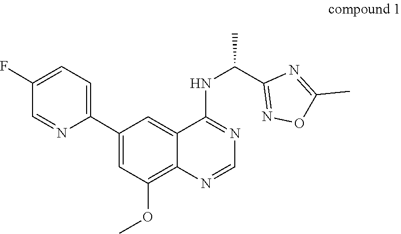 [SMILES: COc1cc(-c2ccc(F)cn2)cc2c(N[C@H](C)c3noc(C)n3)ncnc12]

comprising the steps of:
a) reacting intermediate 2

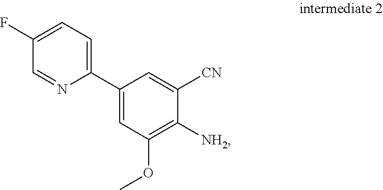 [SMILES: COc1cc(-c2ccc(F)cn2)cc(C#N)c1N]

or a pharmaceutically acceptable salt thereof, with DMF-DMA in the presence of a solvent and an acid;
b) coupling the resulting intermediate 3

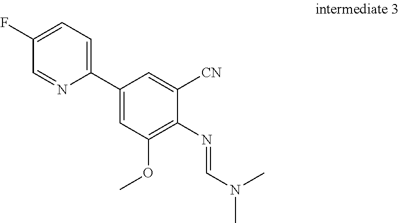 [SMILES: COc1cc(-c2ccc(F)cn2)cc(C#N)c1/N=C/N(C)C]

with (R)-1-(5-methyl-1,2,4-oxadiazol-3-yl)ethan-1-amine (compound 4),

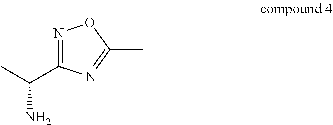

or a pharmaceutically acceptable salt thereof, in the presence of a solvent and a solubilizing agent, thus obtaining compound 1.

### DETAILED DESCRIPTION OF THE INVENTION

Unless defined otherwise, all technical and scientific terms used herein have the same meanings as commonly understood by the skilled in the art.

### Abbreviations

AcOH=Acetic acid; CDI=N,N′-carbonyldiimidazole; DCM=dichloromethane; DIPEA=N,N-Diisopropylethylamine; DMA=dimethylacetamide; DMF=dimethylformamide; DMF-DMA=N,N-Dimethylformamide dimethyl acetal; DMSO-d6=deuterated dimethyl sulfoxide; eq=equivalents; EtOAc=Ethyl acetate; h=hour/s; HCOOH=formic acid; HPLC=High Performance Liquid Chromatography; IPA=isopropyl alcohol; KF=Karl Fischer; mbar=millibar; MeCN=acetonitrile; MeOH=methyl alcohol; 2-MeTHF=2-methyltetrahydrofuran; min=minute/s; (m) mol=(milli) moles; NBS=N-bromosuccinimide; NMR=nuclear magnetic resonance; P=pressure; Pd(dppf)Cl₂=Pd-146=[1,1′-Bis(diphenylphosphino)ferrocene]dichloropalladium(II); Pd-170=chloro(crotyl)(2-dicyclohexylphosphino-2′,4′,6′-triisopropyl-1,1′-biphenyl) palladium(II); Pd black=palladium black; Pd/C=palladium on carbon; KOAc=potassium acetate; CsOAc=cesium acetate; POCl₃=phosphorus (V) oxychloride; NaHCO₃=sodium bicarbonate; NaCl=sodium chloride; Na₂SO₄=sodium sulfate; NaOH=sodium hydroxide; rT=room temperature; T=temperature; TBAHSO₄=tetrabutylammonium hydrogensulfate; THF=tetrahydrofuran; t_{R}=Retention time; UPLC=Ultra Performance Liquid Chromatography; Pd(Ph₃)₂Cl₂=palladium triphenylphosphine dichloride; PdSPhos-G2=Chloro(2-dicyclohexylphosphino-2′,6′-dimethoxy-1,1′-biphenyl)[2-(2′-amino-1,1′-biphenyl)]palladium(II)

### Definitions

The term “compound 1” refers to (R)-6-(5-fluoropyridin-2-yl)-8-methoxy-N-(1-(5-methyl-1,2,4-oxadiazol-3-yl)ethyl)quinazolin-4-amine. It is to be understood that besides the compound 1 showing a preferred (R) configuration, the racemic form and enantiomer (S) are encompassed within the scope of the present invention.

The term “solubilizing agent” refers to a substance which is added to the reaction mixture in step b) and favours the solubilization of compound 4, i.e. (R)-1-(5-methyl-1,2,4-oxadiazol-3-yl)ethan-1-amine.

The term “under reflux” refers to an arrangement in which a reaction is carried out in a boiling solvent with the vapour being condensed and returned to the reaction vessel. The heating needed to reach the suitable temperature depends on the boiling point of the solvent used in the reaction.

The term “about” is to be construed as modifying a term or value such that it is not an absolute. Such term will be defined by the circumstances. This includes, at the very least, the degree of expected experimental error, technique error and instrument error for a given technique used to measure a value.

The term “room temperature”, abbreviated to rT, means a temperature in the range of about 15° C. to about 25° C., with an average of about 23° C.

The term “pharmaceutically acceptable salts”, as used herein, refers to derivatives of compound 1 (or of intermediate 2, or of intermediate 3) wherein such compound is suitably modified by converting the free basic group into the corresponding addition salt with any acid conventionally intended as being pharmaceutically acceptable. Suitable examples of said salts may thus include mineral or organic acid addition salts of basic residues such as amino groups. The salts obtained by reacting the compound, functioning as a base, with an inorganic or organic acid to form a salt comprise, for example, salts of hydrochloric acid, hydrobromic acid, sulfuric acid, phosphoric acid, methane sulfonic acid, camphor sulfonic acid, acetic acid, oxalic acid, maleic acid, fumaric acid, succinic acid and citric acid.

The term “treating”, or “treatment” of a disease state includes: (i) inhibiting the disease state, i.e. arresting the development of the disease state or its clinical symptoms, or (ii) relieving the disease state, i.e. causing temporary or permanent regression of the disease state or its clinical symptoms.

The term “preventing”, or “prevention” of a disease state includes causing the clinical symptoms of the disease state not to develop in a subject that may be exposed to or predisposed to the disease state but does not yet experience or display symptoms of the disease state. For example, treating or preventing a respiratory disease or disorder includes treating or preventing the symptoms the disorder such as cough and/or urge to cough associated with a respiratory disease.

The term “therapeutically effective amount” means an amount of a compound that, when administered to a subject for treating a disease state, is sufficient to affect such treatment for the disease state. The “therapeutically effective amount” will vary depending on the compound, disease state being treated, the severity or the disease treated, the age and relative health of the subject, the route, and form of administration.

A straightforward, efficient and large scale synthesis of compound 1 has been devised, wherein the quinazoline bicyclic core is formed at the very last step, which is a coupling-rearrangement reaction between (E)-N′-(2-cyano-4-(5-fluoropyridin-2-yl)-6-methoxyphenyl)-N,N-dimethylformimidamide (intermediate 3) and (R)-1-(5-methyl-1,2,4-oxadiazol-3-yl)ethan-1-amine (compound 4). Such reaction is fast and clean and leads to compound 1 in very good yield and with a high purity, as it may be appreciated in the experimental part of the present application.

Intermediate 3 may be obtained from intermediate 2 and can undergo the coupling-rearrangement reaction to yield compound 1 either after isolation (two-step process) or directly, i.e. without prior isolation, after a simple and fast work-up procedure (one-pot process). Thus, both intermediate 2 and intermediate 3 are key intermediates in the preparation of compound 1 according to the invention.

Typically, intermediate 2 is suspended in a solvent followed by addition of DMF-DMA in the presence of an acid, such as acetic acid. After the formation of the formamidine intermediate 3, such compound may be isolated or may react directly with compound 4, which is a compound known in the art, in the presence of a solubilizing agent.

Intermediate 2 was best prepared by reacting 2-amino-5-bromo-3-methoxybenzonitrile with bis(pinacolato)diboron in the presence of a Pd catalyst and a base and subsequent reaction with 2-bromo-5-fluoropyridine, for instance as described in Example 2. The reaction may be carried out in a solvent selected from the group consisting of THF, dioxane, toluene, 2-MeTHF, EtOAC, IPA and isopropyl acetate. Preferably the solvent is THF. The preferred Pd catalyst is Pd(dppf)Cl₂, but other Pd catalysts known in the art may be used, for instance Pd(Ph₃)₂Cl₂, PdSPhos-G2, Pd-170, Pd black and Pd/C 5%, in an amount range of about 2-8%. The base may be selected for instance from the group consisting of KOAc, CsOAc and potassium 2-ethyl hexanoate, but other bases known in the art may be used. In a preferred embodiment KOAc is used, together with ether 18-crown-6 which helps in the dissolution. Bis(pinacolato)diboron is used in excess, typically in an amount of about 1.25-1.7 eq relative to the benzonitrile starting material. When the formation of the intermediate pinacol boronate is complete, after refluxing for at least 2 h, typically from 2 to 6 h, the subsequent step involves the reaction with 2-bromo-5-fluoropyridine, in slight excess, under basic aqueous conditions under reflux, for a time range of at least 4 h, typically from 4 h to 12 h, until completion.

The starting material 2-amino-5-bromo-3-methoxybenzonitrile may be prepared as described in the art, or for instance as described in Example 1.

Accordingly, the invention provides 2-amino-5-(5-fluoropyridin-2-yl)-3-methoxy benzonitrile (intermediate 2) or a pharmaceutically acceptable salt thereof.

The invention also provides a process for the preparation of intermediate 2 comprising the step of reacting 2-amino-5-bromo-3-methoxybenzonitrile with bis(pinacolato)diboron in the presence of a Pd catalyst and a base and subsequent reaction with 2-bromo-5-fluoropyridine.

The invention further provides the use of intermediate 2 for the preparation of compound 1, or a pharmaceutically acceptable salt thereof.

The invention also provides (E)-N′-(2-cyano-4-(5-fluoropyridin-2-yl)-6-methoxyphenyl)-N,N-dimethylformimidamide (intermediate 3) or a pharmaceutically acceptable salt thereof.

Further, the invention provides a process for the preparation of intermediate 3 comprising the step of:
a) reacting intermediate 2, or a pharmaceutically acceptable salt thereof, with DMF-DMA in the presence of a solvent and an acid.

The invention further provides the use of intermediate 3 for the preparation of compound 1.

Accordingly, the present invention provides a process for the preparation of compound 1, or a pharmaceutically acceptable salt thereof, comprising the steps of:
a) reacting intermediate 2, or a pharmaceutically acceptable salt thereof, with DMF-DMA in the presence of a solvent and an acid;
b) coupling the resulting intermediate 3 with (R)-1-(5-methyl-1,2,4-oxadiazol-3-yl)ethan-1-amine (compound 4), or a pharmaceutically acceptable salt thereof, in the presence of a solvent and a solubilizing agent, thus obtaining compound 1.

As to step a), DMF-DMA may be used in excess, spanning from a small to a large excess. In a preferred embodiment, DMF-DMA is used in an amount range of 1.05-1.5 eq, most preferably in the range 1.3-1.4 eq, relative to intermediate 2. The solvent may be selected from the group consisting of MeCN, 2-MeTHF, THF, EtOAc, IPA and acetic acid, or mixtures thereof. Preferably, the solvent is selected from MeCN and 2-MeTHF. Acetic acid is the preferred acid when not already used as the solvent. More preferably, the acid is used in a catalytic amount, for instance in the range 0.04-0.15 eq. Even more preferably, acetic acid is used in a catalytic amount in the range 0.045-0.08 eq. Step a) is carried out under reflux for a time range of at least 1 h, typically from 1 h to 5 h, until completion.

As to step b), compound 4 is typically used in an amount range of 1.1-1.5 eq relative to the amount of intermediate 2 used in step a) or to the amount of intermediate 3, when that is weighed after isolation. Further small amounts of compound 4 may be used to drive the reaction to completion. Preferably, compound 4 is used as hydrochloride salt. The solvent may be selected from the group consisting of MeCN, 2-MeTHF, THF, EtOAc, IPA and acetic acid. Preferably, the solvent is selected from MeCN and 2-MeTHF. The solubilizing agent is preferably selected from sodium trifluoromethanesulfonate and sodium mesylate, and is preferably used in an amount range of 0.9-1.1 eq relative to compound 4, more preferably 1 eq. Step b) is carried out under reflux for a time range of at least 1 h, typically from 1 h to 5 h, until completion.

In a preferred embodiment, step a) and step b) are performed without the isolation of intermediate 3, as a one-pot process. In case such one-pot process is performed in acetonitrile, the reaction temperature is particularly mild, being 82° C. the boiling point of acetonitrile and being the reaction carried out under reflux, yet allowing for a fast coupling-rearrangement reaction.

However, intermediate 3 may be isolated as a solid product with good HPLC purity from the reaction mixture of step a) with a simple work-up procedure, for instance as described in Example 4, before undergoing transformation in step b).

The present invention also provides a process for the preparation of compound 1, further comprising the preparation of intermediate 2 according to the step of reacting 2-amino-5-bromo-3-methoxybenzonitrile with bis(pinacolato)diboron in the presence of a Pd catalyst and a base and subsequent reaction with 2-bromo-5-fluoropyridine.

In one preferred embodiment, the present invention provides a process for the preparation of compound 1 according to the following:

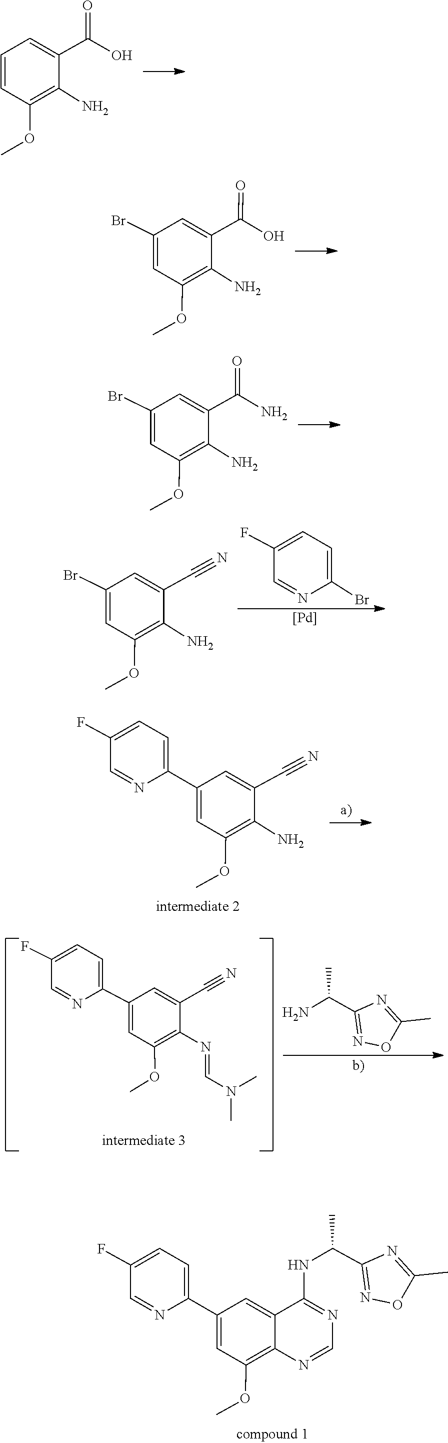

Such process, as depicted in Scheme 1, provides compound 1 in 5 steps starting from 2-amino-5-bromo-3-methoxybenzoic acid with an overall yield of 60%. Such process according to the invention is convergent, highly efficient, economical, and has been carried out in large scale, leading to compound 1 with excellent purity, without the need for any intermediate or final chromatographic purification.

Compound 1 as prepared by the process according to the invention may be used in pharmaceutical compositions for medical use, in particular for the treatment of chronic cough.

Accordingly, the present invention provides a pharmaceutical composition comprising compound 1, or a pharmaceutically acceptable salt thereof, in admixture with one or more pharmaceutically acceptable carrier or excipient, either alone or in combination with one or more further active ingredient.

In one aspect, the invention provides compound 1, or a pharmaceutically acceptable salt thereof, obtained as described above according to the invention, for use as a medicament.

In a further aspect, the invention provides the use of compound 1, or a pharmaceutically acceptable salt thereof, obtained as described above according to the invention, in the manufacture of a medicament for the treatment of disorders associated with P2X₃ receptors mechanism, preferably for the treatment of respiratory diseases.

Preferably, the invention provides compound 1, or a pharmaceutically acceptable salt thereof, obtained as described above according to the invention, for use in the prevention and/or treatment of respiratory diseases, preferably cough, sub-acute or chronic cough, treatment-resistant cough, idiopathic chronic cough, post-viral cough, iatrogenic cough, asthma, idiopathic pulmonary fibrosis (IPF), chronic obstructive pulmonary disease (COPD) and cough associated with respiratory diseases such as COPD, asthma and bronchospasm.

More preferably, the invention provides compound 1, or a pharmaceutically acceptable salt thereof, obtained as described above according to the invention, for use in the prevention and/or treatment of chronic cough and cough associated with respiratory diseases such as COPD, asthma and bronchospasm.

The invention also provides a method for the prevention and/or treatment of disorders associated with P2X₃ receptors mechanisms, said method comprising administering to a patient in need of such treatment a therapeutically effective amount of compound 1, or a pharmaceutically acceptable salt thereof, obtained as described above according to the invention.

In particular the invention refers to a method for the prevention and/or treatment, wherein the disorder is cough, sub-acute or chronic cough, treatment-resistant cough, idiopathic chronic cough, post-viral cough, iatrogenic cough, asthma, idiopathic pulmonary fibrosis (IPF), chronic obstructive pulmonary disease (COPD) and cough associated with respiratory diseases such as COPD, asthma and bronchospasm, wherein said method comprises the administration of a proper amount of compound 1, or a pharmaceutically acceptable salt thereof, obtained as described above according to the invention, to a patient in need thereof. In a further preferred embodiment, the disorder is chronic cough.

The methods of treatment of the invention comprise administering a safe and effective amount of compound 1, or a pharmaceutically acceptable salt thereof, obtained as described above according to the invention, to a patient in need thereof. As used herein, “safe and effective amount” in reference to compound 1, or a pharmaceutically acceptable salt thereof, obtained as described above according to the invention, or other pharmaceutically-active agent, means an amount of the compound sufficient to treat the patient's condition but low enough to avoid serious side effects, such amount being nevertheless routinely determined by the skilled artisan. Compound 1, or a pharmaceutically acceptable salt thereof, obtained as described above according to the invention, may be administered once or according to a dosing regimen wherein a number of doses are administered at varying intervals of time for a given period of time. Typical daily dosages may vary depending upon the particular route of administration chosen.

The invention also provides pharmaceutical compositions of the compound 1, or a pharmaceutically acceptable salt thereof, obtained as described above according to the invention, in a mixture with one or more pharmaceutically acceptable carrier or excipient, for example those described in Remington's Pharmaceutical Sciences Handbook, XVII Ed., Mack Pub., N.Y., U.S.A.

Administration of the compound 1, or a pharmaceutically acceptable salt thereof, obtained as described above according to the invention, may be accomplished according to patient needs, for example, orally, nasally, parenterally (subcutaneously, intravenously, intramuscularly, intrasternally and by infusion) and by inhalation.

Preferably, compound 1, or a pharmaceutically acceptable salt thereof, obtained as described above according to the invention, may be administered orally or by inhalation.

Various solid oral dosage forms may be used for administering compound 1, or a pharmaceutically acceptable salt thereof, obtained as described above according to the invention, including such solid forms as tablets, gelcaps, capsules, caplets, granules, lozenges and bulk powders. Compound 1, or a pharmaceutically acceptable salt thereof, obtained as described above according to the invention, may be administered alone or combined with various pharmaceutically acceptable carriers, diluents (such as sucrose, mannitol, lactose, starches) and known excipients, including suspending agents, solubilizers, buffering agents, binders, disintegrants, preservatives, colorants, flavorants, lubricants and the like. Time release capsules, tablets and gels are also advantageous in administering the compounds of the invention.

Preferably, compound 1, or a pharmaceutically acceptable salt thereof, obtained as described above according to the invention, is administered orally, more preferably in the form of tablets.

Various liquid oral dosage forms may also be used for administering compound 1, or a pharmaceutically acceptable salt thereof, obtained as described above according to the invention, including aqueous and non-aqueous solutions, emulsions, suspensions, syrups, and elixirs. Such dosage forms can also contain suitable known inert diluents, such as water, and suitable known excipients, such as preservatives, wetting agents, sweeteners, flavorants, as well as agents for emulsifying and/or suspending compound 1. Compound 1, or a pharmaceutically acceptable salt thereof, obtained as described above according to the invention, may be injected, for example, intravenously, in the form of an isotonic sterile solution.

For the treatment of the diseases of the respiratory tract, compound 1, or a pharmaceutically acceptable salt thereof, obtained as described above according to the invention, may be administered by inhalation. Inhalable preparations include inhalable powders, propellant-containing metering aerosols or propellant-free inhalable formulations.

For administration as a dry powder, single- or multi-dose inhalers known in the art may be utilized. In that case the powder may be filled in gelatine, plastic or other capsules, cartridges or blister packs or in a reservoir.

A diluent or carrier chemically inert to the compounds of the invention, e.g. lactose or any other additive suitable for improving the respirable fraction, may be added to the powdered compound 1.

Inhalation aerosols containing propellant gas such as hydrofluoroalkanes may contain compound 1, or a pharmaceutically acceptable salt thereof, obtained as described above according to the invention, either in solution or in dispersed form. The propellant-driven formulations may also contain other ingredients such as co-solvents, stabilizers and optionally other excipients.

The propellant-free inhalable formulations comprising compound 1, or a pharmaceutically acceptable salt thereof, obtained as described above according to the invention, may be in the form of solutions or suspensions in an aqueous, alcoholic or hydroalcoholic medium and they may be delivered by jet or ultrasonic nebulizers known from the prior art or by soft-mist nebulizers.

The compound 1, or a pharmaceutically acceptable salt thereof, obtained as described above according to the invention, may be administered as the sole active agent or in combination with other pharmaceutical active ingredients.

Preferably, the compound 1, or a pharmaceutically acceptable salt thereof, obtained as described above according to the invention, may be combined with therapeutic agents or active ingredients useful for the treatment of disease which are related to or mediated by P2X3 receptor.

The dosages of the compound 1, or a pharmaceutically acceptable salt thereof, obtained as described above according to the invention, depend upon a variety of factors including, among others, the particular disease to be treated, the severity of the symptoms, the route of administration, and the like.

The invention is also directed to a device comprising a pharmaceutical composition comprising compound 1, or a pharmaceutically acceptable salt thereof, obtained as described above according to the invention, in form of a single- or multi-dose dry powder inhaler or a metered dose inhaler.

### EXPERIMENTAL PART

IUPAC Chemical Names of the compounds were generated with Structure-To-Name tool of PerkinElmer ChemDraw Professional application (v. 20.0.0.41).

Where the preparation of starting materials or other reagents is not described, these are commercially available, known in the literature, or readily obtainable by those skilled in the art using standard procedures. All solvents were purchased from commercial sources and were used without additional purification.

### General Characterization Details

### NMR

¹H Nuclear magnetic resonance spectroscopy was carried out using a Varian and Bruker instrument operating at 400 and 600 MHz respectively using the stated solvent at around room temperature unless otherwise stated. In all cases, NMR data were consistent with the proposed structures. Characteristic chemical shifts (δ) are given in parts-per-million using conventional abbreviations for designation of major peaks: e.g. s, singlet; d, doublet; t, triplet; q, quartet; quin, quintet; dd, doublet of doublets; dt, doublet of triplets; m, multiplet; br, broad.

### LC Analytical Methods

Method 1 (UPLC): Acquity CSH C18 column 50 mm×2.1 mm 1.7 μm, maintained at 40° C.; Mobile Phase: Eluent B (MeCN/water 95:5+0.05% HCOOH) in Eluent A (water/MeCN 95:5+0.05% HCOOH) from 1% to 99.9% within 2 min or 4 min. Flow rate: 1 mL/min. Wavelength: 210-400 nm DAD.

Method 2 (HPLC): Agilent Poroshell 120 EC-C18 100×2.1 mm, 1.9 m maintained at 40° C.; mobile phase: Eluent B (MeCN) in Eluent A (ammonium acetate buffer 0.01 M pH=7). Flow rate: 1 mL/min, run time=20 min.

Gradient

| Time (min) | Eluent A (%) | Eluent B (%) |
|---|---|---|
| 0 | 98 | 2 |
| 15 | 40 | 60 |
| 17 | 40 | 60 |
| 18 | 98 | 2 |
| 20 | 98 | 2 |

Method 3 (Chiral HPLC): (S,S)-Whelk-O 1 1.8 μm Kromasil, 100*4.6 mm, 1.8 μm maintained at 35° C.; mobile phase: Eluent B (EtOH/THF [85:15]5 mM TBAHSO₄) in Eluent A (water 5 mM TBAHSO₄). Flow rate: 1 mL/min. Run time=35 min. Isocratic conditions.

### Procedures

### Example 1: Preparation of 2-amino-5-bromo-3-methoxybenzonitrile

### Step 1—2-amino-5-bromo-3-methoxybenzoic acid

NBS (1.35 kg, 7.6 mol, 1.10 eq) was added portion-wise to a solution of 2-amino-3-methoxybenzoic acid (1.15 kg, 6.9 mol, 1.0 eq) in DMF (6.9 L) by keeping the temperature lower then 10° C., and the reaction mixture was first stirred at 0-10° C. for 1 h and then at 20° C. for 2 h. The reaction mixture was poured into water (18.4 L) at T<25° C. and stirred for 30 min. The solid was filtered and washed with water (6.9 L). The filter cake was dried under vacuum at 60° C. to afford 2-amino-5-bromo-3-methoxybenzoic acid as a brownish powder (1.65 kg; yield 97%; UPLC purity=99.1% area % according to Method 1).

¹H NMR (DMSO-d₆, 600 MHz) δ 7.41 (d, 1H, J=2.20 Hz) 7.07 (d, 1H, J=2.20 Hz) 3.83 (s, 3H)

### Step 2—2-amino-5-bromo-3-methoxybenzamide

CDI (1.24 kg, 7.6 mmol, 1.06 eq) was added portion-wise to a solution of 2-amino-5-bromo-3-methoxybenzoic acid (1.768 kg, 7.2 mmol, 1.00 eq) in THE (8.8 L) at 10-20° C. The reaction mixture was stirred at 20° C. for at least 1 h. Then 30 wt % aqueous ammonia (545 mL, 28.8 mmol, 4.00 eq) was added dropwise at 10-20° C. The reaction was stirred for 2 h at 20° C. The reaction was concentrated while adding water (18 L) and the mixture was cooled down at 15° C. The aqueous layer was extracted with 2-MeTHF (200 mL), and the combined organic layers were washed with water (80 mL) and brine (80 mL), then concentrated up to 3.5-4 L. The mixture was filtered and washed with water. The filter cake was dried under vacuum at 50° C. to afford 2-amino-5-bromo-3-methoxybenzamide brownish powder (1.51 kg; yield 86%; UPLC purity=97.5% area % according to Method 1).

¹H NMR (DMSO-d₆, 400 MHz) δ 3.81 (s, 3H), 6.41 (s, 2H), 7.00 (d, 1H, J=1.97 Hz), 7.19 (br s, 1H), 7.40 (d, 1H, J=1.75 Hz), 7.83 (br s, 1H)

### Step 3—2-amino-5-bromo-3-methoxybenzonitrile

To a solution of 2-amino-5-bromo-3-methoxybenzamide (1.4 kg, 5.7 mol, 1.0 eq) in DCM (11.1 L) DIPEA (1.842 kg, 14.2 mol, 2.5 eq) was added at 5° C. Then POCl₃ (939.3 g, 6.84 mol, 1.2 eq) was added dropwise at T<30° C. The mixture reaction was stirred at 30-35° C. for 2 h. At the end of the reaction, water (5.6 L) was added dropwise and the mixture was stirred for 15 min. Then the aqueous phase was separated, and the organic phase was washed twice with 10% NaHCO₃ aqueous (4.2 L), 5% NaCl aqueous (2.8 L) and dried with Na₂SO₄. The organic phase was filtered through a pad of silica gel (0.75 w/w) and eluted with DCM (4.2 L). The collected organic phase was concentrated under vacuum, the obtained residue was dissolved in MeOH (6.9 L) at 50-60° C., and the mixture was stirred until the residue was completely dissolved. The mixture was concentrated up to 3.5-4 L and water (3.5 L) was added dropwise at 50-60° C. Then the mixture was stirred at 5° C. and filtered. The filter cake was dried under vacuum at 50° C. to afford 2-amino-5-bromo-3-methoxybenzonitrile as dark red powder (1.17 kg; yield 91%; UPLC purity=99.0% area % according to Method 1).

¹H NMR (DMSO-d₆, 600 MHz) δ 7.22 (d, 1H, J=2.2 Hz), 7.15 (d, 1H, J=1.9 Hz), 5.92 (s, 2H), 3.84 (s, 3H)

### Example 2: Preparation of 2-amino-5-(5-fluoropyridin-2-yl)-3-methoxybenzonitrile (Intermediate 2)

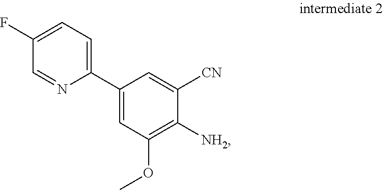 [SMILES: COc1cc(-c2ccc(F)cn2)cc(C#N)c1N]

2-amino-5-bromo-3-methoxybenzonitrile (3.5 kg; 15.4 mol; 1 eq), potassium acetate (4.9 kg; 49.9 mol; 3.20 eq) and ether 18-crown-6 (2.03 kg; 7.69 mol; 0.50 eq) were suspended in THE (35 L). The water content (KF test) of the mixture was tested in order to avoid the consumption of bis(pinacolato)diboron: in case it was found to be higher than 0.5%, the mixture was concentrated to reach the target KF value. Bis(pinacolato)diboron (5.46 kg; 21.5 mol; 1.4 eq) was added. The mixture was vigorously bubbled with nitrogen for at least 30 min. Then Pd(dppf)Cl₂ (i.e. Pd-146, Johnson Matthey, 52.5 g; 0.07 mol; 0.0046 eq) was added and 10 cycles of vacuum/nitrogen were performed. The mixture was then stirred under reflux temperature for at least 2 h.

A solution of potassium carbonate (K₂CO₃, 6.8 kg; 14.8 mol; 3.20 eq) in water (30.4 L) was cautiously added to the previous solution and stirred for at least 60 min. Then 2-bromo-5-fluoropyridine (3.5 kg; 20.1 mol; 1.3 eq) was dissolved in THE (7 L) and added to the reaction mixture after degassing with nitrogen. Fresh Pd-146 (224 g; 0.31 mol; 0.02 eq) was added and the resulting mixture was stirred under reflux for 6-8 h.

The mixture was then cooled down to 55-65° C., the stirring was stopped and the aqueous phase discharged. The collected organic phase was washed with 10% brine three times (10 L each wash). All the obtained aqueous phases were discharged. The organic phase was transferred in a flask and treated with charcoal (350 g) followed by filtration through a celite pad. The filtered solution was concentrated under atmosphere pressure up to 10-14 L; IPA (14 L) was added keeping the temperature at 55-65° C. The suspension was stirred at this temperature for at least 60 min. Then it was cooled down to −5 to 5° C. in at least 2 h and finally kept at −5 to 5° C. under stirring for at least 2 h. The suspension was filtered and the cake was washed with a mixture of water/IPA 90/10 (17.5 L) and finally with water (16.5 L) affording a purplish powder of the title compound (intermediate 2), which was dried at 40-50° C., P<100 mbar, for at least 24 h (3.3 Kg, yield 88.7%; UPLC purity=99.5% area % according to Method 1).

¹H NMR (DMSO-d₆, 600 MHz) δ 8.58 (d, 1H, J=3.1 Hz), 8.04 (dd, 1H, J=4.2, 8.9 Hz), 7.7-7.8 (m, 2H), 7.73 (d, 1H, J=1.9 Hz), 6.04 (s, 2H), 3.92 (s, 3H)

### Example 3: Preparation of (R)-6-(5-fluoropyridin-2-yl)-8-methoxy-N-(1-(5-methyl-1,2,4-oxadiazol-3-yl)ethyl)quinazolin-4-amine (compound 1)—One-pot process

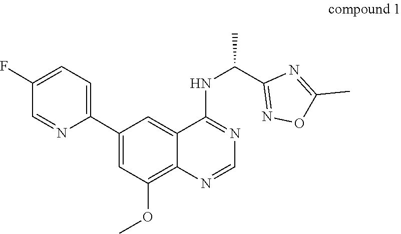 [SMILES: COc1cc(-c2ccc(F)cn2)cc2c(N[C@H](C)c3noc(C)n3)ncnc12]

2-amino-5-(5-fluoropyridin-2-yl)-3-methoxybenzonitrile (intermediate 2.9 kg; 37 mol; 1 eq) was suspended in MeCN (52.5 L) followed by the addition of DMF-DMA (6.7 L; 50.6 mol; 1.4 eq) and catalytic glacial acetic acid (171 mL; 3 mol; 0.081 eq). The resulting mixture was heated under reflux temperature for at least 1 h.

Water (666 mL) was added to the mixture and stirred under reflux for at least 30 min. Then a further amount of MeCN (27.5 L) was added and the mixture was concentrated up to 60-80 L. The mixture was cooled down to 50-60° C. and sodium trifluoromethanesulfonate (6.4 kg; 37.1 mol; 1 eq) and (R)-1-(5-methyl-1,2,4-oxadiazol-3-yl)ethan-1-aminium hydrochloride (7.56 kg; 46.2 mol; 1.25 eq, prepared similarly to what described in WO2011162835, Example 3, by starting from (2R)-2-[(tert-butoxycarbonyl)amino]propanoic acid and cleaving the Boc group with HCl in the last step) were added. The resulting mixture was heated under reflux under nitrogen for at least 2 h and progression was checked by HPLC. The mixture was concentrated under vacuum up to 36-45 L. Then the mixture was cooled down to 55-65° C. and water (54 L) was added and the suspension was stirred at 55-65° C. for at least 30 min. After lowering the temperature to 15-25° C., NaOH 30% was added (13.5 L) and stirred for at least 1 h. The solid was filtered and washed with a mixture of water/IPA 80:20 twice (2×45 L) and finally with water (45 L) affording a brownish powder of the title compound 1, which was dried at 40-50° C., P<100 mbar, for at least 24 h (12.73 kg, yield 90.5%; HPLC purity=99.9% area according to Method 2; chiral HPLC purity=100% area % according to Method 3).

¹H NMR (DMSO-d₆, 600 MHz) δ 8.7-8.8 (m, 2H), 8.58 (d, 1H, J=1.6 Hz), 8.46 (s, 1H), 8.30 (dd, 1H, J=4.4, 9.0 Hz), 8.0-8.0 (m, 2H), 5.77 (quin, 1H, J=7.2 Hz), 4.01 (s, 3H), 2.57 (s, 3H), 1.69 (d, 3H, J=7.2 Hz)

### Example 4: Preparation of (R)-6-(5-fluoropyridin-2-yl)-8-methoxy-N-(1-(5-methyl-1,2,4-oxadiazol-3-yl)ethyl)quinazolin-4-amine (Compound 1)—Two-step process

### Step 1—(E)-N′-(2-cyano-4-(5-fluoropyridin-2-yl)-6-methoxyphenyl)-N,N-dimethylformimidamide (Intermediate 3)

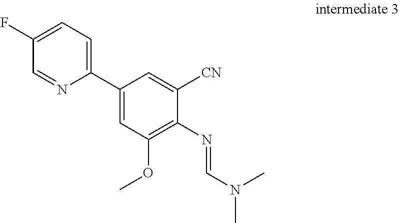 [SMILES: COc1cc(-c2ccc(F)cn2)cc(C#N)c1/N=C/N(C)C]

2-amino-5-(5-fluoropyridin-2-yl)-3-methoxybenzonitrile (intermediate 2, 20 g, 82 mmol) was suspended in 130 mL of MeCN. The reaction mixture was added with DMF-DMA (15 mL, 112 mmol, 1.36 eq) and glacial acetic acid (0.25 mL, 4.5 mmol, 0.05 eq) and the mixture refluxed under nitrogen for 2 h; a fresh amount of DMF-DMA (1.5 mL, 11 mmol, 0.14 eq) and of glacial acetic acid (0.1 mL, 1.7 mmol, 0.02 eq) were added and the reaction mixture stirred under reflux for 1 h. Water (10 mL) was added and the reaction mixture was cooled down to rT, the reaction mixture stirred 16 h at rT and the organic solvent removed under reduced pressure; the residue rinsed with 150 mL of water, the resulting suspension stirred at rT and the solid filtered off under reduced pressure and washed with water (50 mL), affording a brownish powder of the title compound (intermediate 3), which was dried at 40-45° C., P<100 mbar, for at least 16 h (23.2 g, yield 95%; UPLC purity=98.5% area % according to Method 1).

¹H NMR (DMSO-d₆, 600 MHz) δ 8.64 (d, 1H, J=2.9 Hz), 8.14 (dd, 1H, J=4.3, 8.9 Hz), 8.04 (s, 1H), 7.89 (d, 1H, J=1.8 Hz), 7.86 (d, 1H, J=1.8 Hz), 7.83 (dt, 1H, J=3.1, 8.7 Hz), 3.87 (s, 3H), 3.06 (s, 3H), 2.99 (s, 3H)

### Step 2—(R)-6-(5-fluoropyridin-2-yl)-8-methoxy-N-(1-(5-methyl-1,2,4-oxadiazol-3-yl)ethyl)quinazolin-4-amine (Compound 1)

1.5 g (5.0 mmol) of N′-(2-cyano-4-(5-fluoropyridin-2-yl)-6-methoxyphenyl)-N,N-dimethylformimidamide were suspended in 15 mL of MeCN along with 1 g (6.5 mmol, 1.3 eq) of (R)-1-(5-methyl-1,2,4-oxadiazol-3-yl)ethan-1-aminium hydrochloride (prepared as described in Example 3) and 1.1 g (6.5 mmol, 1.3 eq) of sodium trifluoromethanesulfonate and the reaction mixture was stirred under reflux under nitrogen for 2 h. The reaction mixture was then cooled down at rT, the organic solvent removed under reduced pressure and the residue rinsed with 15 mL of water; the suspension was stirred at rT and the brownish solid filtered off under reduced pressure, redissolved in refluxing MeOH and treated with 0.09 g of active charcoal, filtering the hot solution over a celite pad and washing with MeOH. The solution was concentrated to 15 mL under reduced pressure, stirred at 20° C. for 16 h and the solid filtered off under reduced pressure, affording a brownish powder of the title Compound 1, which was dried at 40-50° C., P<100 mbar, for at least 24 h (1.45 g, yield 76%; UPLC purity=98.6% area % according to Method 1).

1: A compound, 2-amino-5-(5-fluoropyridin-2-yl)-3-methoxybenzonitrile, having the formula: 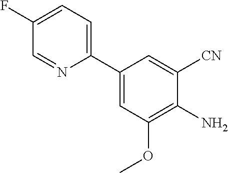 [SMILES: COc1cc(-c2ccc(F)cn2)cc(C#N)c1N]
or a pharmaceutically acceptable salt thereof.

2: A process for the preparation of (R)-6-(5-fluoropyridin-2-yl)-8-methoxy-N-(1-(5-methyl-1,2,4-oxadiazol-3-yl)ethyl)quinazolin-4-amine or a pharmaceutically acceptable salt thereof, comprising reacting the compound or pharmaceutically acceptable salt thereof according to claim 1 with DMF-DMA in the presence of a solvent and an acid.

3: A process for the preparation of the compound or pharmaceutically acceptable salt thereof according to claim 1, comprising reacting 2-amino-5-bromo-3-methoxybenzonitrile with bis(pinacolato)diboron in the presence of a Pd catalyst and a base and subsequent reaction with 2-bromo-5-fluoropyridine.

4: A compound, (E)-N′-(2-cyano-4-(5-fluoropyridin-2-yl)-6-methoxyphenyl)-N,N-dimethylformimidamide, having the formula: 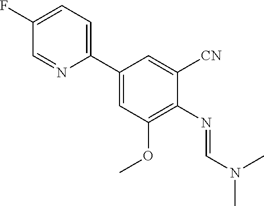 [SMILES: COc1cc(-c2ccc(F)cn2)cc(C#N)c1/N=C/N(C)C]
or a pharmaceutically acceptable salt thereof.

5: A process for the preparation of (R)-6-(5-fluoropyridin-2-yl)-8-methoxy-N-(1-(5-methyl-1,2,4-oxadiazol-3-yl)ethyl)quinazolin-4-amine or a pharmaceutically acceptable salt thereof, comprising coupling the compound or pharmaceutically acceptable salt thereof according to claim 4 with (R)-1-(5-methyl-1,2,4-oxadiazol-3-yl)ethan-1-amine or a pharmaceutically acceptable salt thereof in the presence of a solvent and a solubilizing agent.

6: A process for the preparation of the compound or pharmaceutically acceptable salt thereof according to claim 4, comprising:
a) reacting 2-amino-5-(5-fluoropyridin-2-yl)-3-methoxybenzonitrile, having the formula: 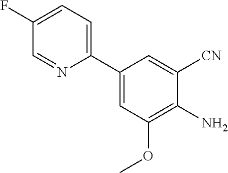 [SMILES: COc1cc(-c2ccc(F)cn2)cc(C#N)c1N]
or a pharmaceutically acceptable salt thereof, with DMF-DMA in the presence of a solvent and an acid.

7: A process for the preparation of (R)-6-(5-fluoropyridin-2-yl)-8-methoxy-N-(1-(5-methyl-1,2,4-oxadiazol-3-yl)ethyl)quinazolin-4-amine, having the formula: 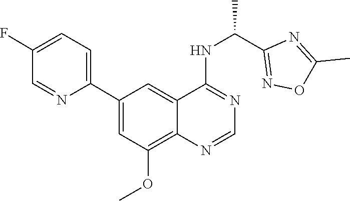 [SMILES: COc1cc(-c2ccc(F)cn2)cc2c(N[C@H](C)c3noc(C)n3)ncnc12]
or a pharmaceutically acceptable salt thereof,
comprising:
a) reacting 2-amino-5-(5-fluoropyridin-2-yl)-3-methoxybenzonitrile, having the formula: 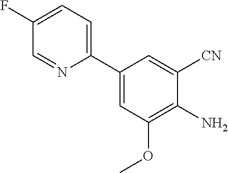 [SMILES: COc1cc(-c2ccc(F)cn2)cc(C#N)c1N]
or a pharmaceutically acceptable salt thereof, with DMF-DMA in the presence of a solvent and an acid; and
b) coupling a resulting intermediate, (E)-N′-(2-cyano-4-(5-fluoropyridin-2-yl)-6-methoxyphenyl)-N,N-dimethylformimidamide, having the formula: 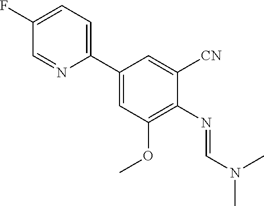 [SMILES: COc1cc(-c2ccc(F)cn2)cc(C#N)c1/N=C/N(C)C]
or a pharmaceutically acceptable salt thereof,
with (R)-1-(5-methyl-1,2,4-oxadiazol-3-yl)ethan-1-amine, having the formula: 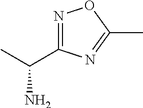
or a pharmaceutically acceptable salt thereof, in the presence of a solvent and a solubilizing agent, thus obtaining (R)-6-(5-fluoropyridin-2-yl)-8-methoxy-N-(1-(5-methyl-1,2,4-oxadiazol-3-yl)ethyl)quinazolin-4-amine or a pharmaceutically acceptable salt thereof.

8: The process according to claim 7, wherein the solvent in a) is selected from the group consisting of acetonitrile and 2-methyltetrahydrofuran.

9: The process according to claim 7, wherein the acid is acetic acid.

10: The process according to claim 7, wherein the solvent in b) is selected from the group consisting of acetonitrile and 2-methyltetrahydrofuran.

11: The process according to claim 7, wherein the solubilizing agent in b) is selected from the group consisting of sodium trifluoromethanesulfonate and sodium mesylate.

12: The process according to claim 7, wherein steps a) and b) are performed without isolating the intermediate (E)-N′-(2-cyano-4-(5-fluoropyridin-2-yl)-6-methoxyphenyl)-N,N-dimethylformimidamide or pharmaceutically acceptable salt thereof.

13: The process according to claim 7, further comprising preparing the 2-amino-5-(5-fluoropyridin-2-yl)-3-methoxybenzonitrile or pharmaceutically acceptable salt thereof by reacting 2-amino-5-bromo-3-methoxybenzonitrile with bis(pinacolato)diboron in the presence of a Pd catalyst and a base and subsequent reaction with 2-bromo-5-fluoropyridine.
